(19) United States
(12) Patent Application Publication
(10) Pub. No.: US 2010/0016446 A1
(43) Pub. Date: Jan. 21, 2010

(54) STABLE WATER-BASED TOPICAL PHARMACEUTICAL CREAMS AND METHODS OF MAKING AND USING SAME

(76) Inventors: Sylvia Gonda, Birmingham, AL (US); Matthew A. Gonda, Birmingham, AL (US)

Correspondence Address: Ballard Spahr LLP, SUITE 1000, 999 PEACHTREE STREET, ATLANTA, GA, 30309-3915, US

(21) Appl. No.: 12/506,701

(22) Filed: Jul. 21, 2009

Related U.S. Application Data
(60) Provisional application No. 61/082,459, filed on Jul. 21, 2008.

(51) Int. Cl. A61K 31/04; A61P 9/10; A61P 17/02

(52) U.S. Cl. 514/742

(57) ABSTRACT

Disclosed are stable, water-based topical creams comprising nitroglycerin and suitable for pharmaceutical and/or cosmetic use. Also disclosed are methods for preparing the creams. Also disclosed are methods for treating circulatory disorders, methods for preventing or alleviating insufficient circulation, methods for enhancing localized activity of systemically administered drugs, methods for enhancing circulation, and methods for promoting wound healing by administering the disclosed creams. This abstract is intended as a scanning tool for purposes of searching in the particular art and is not intended to be limiting of the present invention.

### CROSS-REFERENCE TO RELATED APPLICATIONS

This application claims priority to U.S. Provisional Patent Application No. 61/082,459, filed Jul. 21, 2008, which application is incorporated herein by reference in its entirety.

### BACKGROUND

Topical nitroglycerin compositions (e.g., oils, creams, gels) are noted for their vasodilatory effects and have been used to treat a variety of maladies, including microvascular diseases, erectile dysfunction, and female anorgasmia. However, organic-based compositions can provide the active agent in a carrier system that lacks a satisfactory look and feel for the patient for many topical applications. Further, organic-based compositions necessarily expose a patient to organic-based components and implicate environmental issues (e.g., use of organic solvents) during preparation, use, and disposal.

In contrast, water-based creams can be especially effective for topical administration to patients that exhibit symptoms of these maladies. U.S. Pat. No. 5,698,589, for example, discribes such creams. Unfortunately, conventional water-based nitroglycerin creams can have unsatisfactory stability over time and, therefore, decreased shelf-life. Moreover, reformulation of conventional nitroglycerin creams into a formulation that provides a stable cream, thereby extending shelf-life, is not a trivial undertaking, as the relationships between pharmaceutical composition, drug efficacy, active ingredients, and cream stability have not yet been fully understood.

Even further, conventional processes for water-based nitroglycerin cream preparation have been unable to provide straightforward, single-vessel procedures that yield stable, uniform, water-based creams. Therefore, there remains a need for stable, uniform, water-based creams for topical administration of nitroglycerin as well as straightforward methods for making same.

### SUMMARY

In accordance with the purpose(s) of the invention, as embodied and broadly described herein, the invention, in one aspect, relates to stable water-based topical pharmaceutical creams and methods of making and using same.

Disclosed are water-based topical creams comprising nitroglycerin; one or more penetration enhancers; at least about 60% of an aqueous solvent by weight of the cream; and one or more pH-adjusters in an amount sufficient to provide pH of the cream at from about 4 to about 6.

Also disclosed are water-based topical creams comprising nitroglycerin and at least about 60% of an aqueous solvent by weight of the cream, wherein the cream has a six-month accelerated decomposition measurement of at least about 60% nitroglycerin.

Also disclosed are methods for preparing a water-based topical cream, the method comprising the steps of providing a mixture of water, one or more penetration enhancers, and, optionally, one or more preservatives; adding to the mixture one or more thickeners; adding to the mixture one or more emulsifiers; heating the mixture to at least a temperature sufficient to melt or solubilize the one or more thickeners and/or one or more emulsifiers; and adjusting the pH of the mixture to from about 4 to about 6 with a pH-adjuster.

Also disclosed are products produced by the disclosed methods.

Also disclosed are methods of treating a circulatory disorder comprising topically administering to a mammal a therapeutically effective amount of water-based topical cream comprising nitroglycerin and at least about 60% by weight of an aqueous solvent and having a pH of from about 4 to about 6, thereby treating the disorder in the mammal.

Also disclosed are methods of preventing or alleviating insufficient circulation comprising co-administering to a mammal a therapeutically effective amount of water-based topical cream comprising nitroglycerin and at least about 60% of an aqueous solvent by weight of the cream and having a pH of from about 4 to about 6 with a drug having a known side-effect of decreasing circulation, thereby preventing or alleviating the insufficient circulation in the mammal.

Also disclosed are kits comprising a drug having a known side-effect of decreasing circulation and a water-based topical cream comprising nitroglycerin.

Also disclosed are methods of preventing or alleviating insufficient circulation comprising co-administering to a mammal a therapeutically effective amount of water-based topical cream comprising nitroglycerin and at least about 60% of an aqueous solvent by weight of the cream and having a pH of from about 4 to about 6 with a drug known to treat a disorder associated with insufficient circulation, thereby preventing or alleviating the insufficient circulation in the mammal.

Also disclosed are kits comprising a drug known to treat a disorder associated with insufficient circulation and a water-based topical cream comprising nitroglycerin.

Also disclosed are methods of enhancing localized activity of a systemically administered drug comprising topically administering to an external surface area of a mammal a therapeutically effective amount of water-based topical cream comprising nitroglycerin and at least about 60% of an aqueous solvent by weight of the cream; and substantially simultaneously administering a drug having a desired systemic effect, thereby enhancing the desired systemic effect proximate to the external surface area of the mammal.

Also disclosed are kits comprising a drug having a desired systemic effect and a water-based topical cream comprising nitroglycerin.

Also disclosed are methods of enhancing circulation comprising topically administering to an external surface area of a mammal an effective amount of water-based topical cream comprising nitroglycerin and at least about 60% of an aqueous solvent by weight of the cream, thereby increasing circulation in the vasculature proximate to the external surface area of the mammal to a level greater than that of the external surface area prior to administration.

Also disclosed are kits comprising one or more nutrients and a water-based topical cream comprising nitroglycerin.

Also disclosed are methods of promoting wound healing comprising topically administering at or proximate to injured tissue of a mammal an effective amount of water-based topical cream comprising nitroglycerin and at least about 60% of an aqueous solvent by weight of the cream, thereby increasing hemodynamic blood flow into the injured tissue.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

This patent or application file contains at least one drawing executed in color. Copies of this patent or patent application publication with color drawings will be provided by the U.S. Patent and Trademark Office upon request and payment of the necessary fee.

The accompanying figures, which are incorporated in and constitute a part of this specification, illustrate several aspects and together with the description serve to explain the principles of the invention.

FIG. 1 shows a color change assay as a function of pH for 0.6% nitroglycerin aqueous creams under a two-month accelerated decomposition (38-40° C.) study.

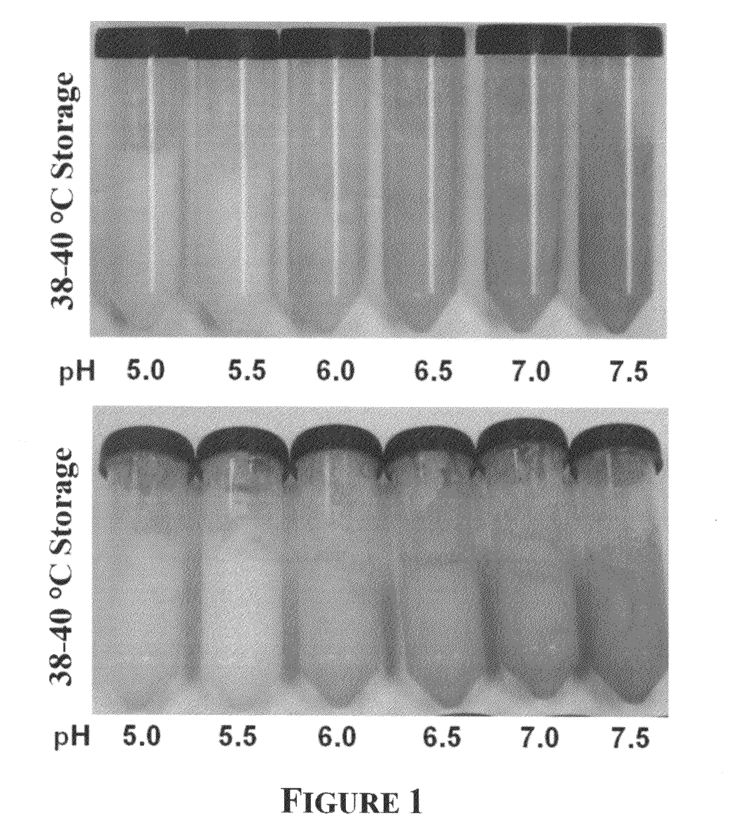

FIG. 2 shows the effect of pH on gel formation as a function of droplet diameter, which is correlated to composition viscosity. The same amount of material taken from each sample was placed on a glass surface, showing the increase of cream firmness (indicated by smaller diameter) with increasing pH.

FIG. 3 shows effect of pH on cream stability as a function of droplet diameter, which is correlated to composition viscosity, for 0.6% nitroglycerin aqueous creams under a five-month accelerated decomposition (38-40° C.) study, compared to analogous formulations held at temperatures of 4-5° C. The same amount of sample was taken from all creams and assays were done at room temperature. Results indicate that all different initial pH creams incubated at 4-5° C., remain firm, while creams incubated at accelerated 38-40° C. temperatures, deteriorate with increasing initial pH, as indicated by the loss in viscosity by the spreading of the droplet.

FIG. 4 shows pH changes in creams containing nitroglycerin prepared at different initial pHs and incubated under accelerated decomposition conditions (38-40° C.) as a function of time. Increasing decomposition of nitroglycerin is associated with increasing pH in cream formulations over time.

FIG. 5 shows a plot of starting pH versus relative viscosity (i.e., each data point is plotted relative to the highest observed). For initial creams containing nitroglycerin (0 months), the creams thicken as pH increases, reaching a maximum viscosity around pH 7. Below pH 4, the mixture is a thick liquid. For nitroglycerin creams incubated at 38-40° C. for 6 months, viscosity is decreased by a larger amount for creams with a higher initial pH. Even though the higher pH creams containing nitroglycerin are initially more viscous, they lose viscosity much faster with incubation at 38-40° C. than lower-pH prepared creams.

FIG. 6 shows the overall effects of incubating a nitroglycerin (NTG) cream at 38-40° C. for 6 months. The three curves represent the percent changes in pH (curve A), percent changes in HPLC nitroglycerin peak area (curve B) and percent changes in viscosity (curve C), as functions of initial pH of cream. All curves are plotted relative to comparative 4-5° C. samples, which are assigned the value of 100%.

FIG. 7 shows an exemplary HPLC chromatogram of sample cream prepared at pH 5.1 and incubated for 4.5 months at 38-40° C. Methyl and Propyl paraben are used in cream as antimicrobials, and ethyl paraben is used as an internal standard.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which may need to be independently confirmed.

### A. Definitions

As used in the specification and the appended claims, the singular forms “a,” “an” and “the” include plural referents unless the context clearly dictates otherwise. Thus, for example, reference to “a cream,” “an ingredient,” or “a drug” includes mixtures of two or more such creams, ingredients, or drugs, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or can not occur, and that the description includes instances where said event or circumstance occurs and instances where it does not.

As used herein, the term “substantially” means that the subsequently described event or circumstance completely occurs or that the subsequently described event or circumstance generally, typically, or approximately occurs. For example, when the specification discloses that method steps are performed substantially simultaneously, a person skilled in the relevant art would readily understand that the steps need not be synchronized. Rather, this term conveys to a person skilled in the relevant art that the method steps can be synchronized, can be overlapping in time, or can be separated by a technically insignificant (e.g., commercially insignificant) amount of time.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. In certain aspects, this term can be synonymous with the language “preventative treatment.”

As used herein, the term “alleviate” or “alleviating” refers to lightening or lessening the severity of a symptom, condition, or disorder. For example, a treatment that reduces the severity of pain in a subject can be said to alleviate pain. It is understood that, in certain circumstances, a treatment can alleviate a symptom or condition without treating the underlying disorder. In certain aspects, this term can be synonymous with the language “palliative treatment.”

As used herein, the term “diagnosed with” a condition refers to having been subjected to a physical examination by a person of skill, for example, a medical doctor (e.g., physician or veterinarian), and found to have the condition. It is also specifically contemplated that a subject (e.g., a mammal, a human) can be identified with such condition.

As used herein, the term “diagnosed with a need for” a treatment refers to having been subjected to a physical examination by a person of skill, for example, a physician, and found to have a condition that can be diagnosed or treated by the treatment. It is also specifically contemplated that a subject (e.g., a mammal, a human) can be identified with a need for such treatment.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “stable,” when referring to a pharmaceutical cream composition, means that the cream can be kept at room temperature for a minimum of six months while retaining at least 90% activity, for example 91% activity, 92% activity, 93% activity, 94% activity, or 95% activity of active component, for example nitroglycerin.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, methanol, isopropanol, polyols (such as glycerol, propylene glycol, polyethylene glycol and the like), carboxymethylcellulose and suitable mixtures thereof, vegetable oils (such as olive oil, light mineral oil, cottonseed oil, castor oil, and the like) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid, chlorhexidine digluconate, and the like. Antioxidants, such as BHT, can be included. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compositions

In one aspect, the invention relates to stable, uniform, water-based topical creams of nitroglycerin. Such topical creams can be useful in stimulating blood flow to tissue containing peripheral nerves in patients suffering from diabetic peripheral neuropathy, Raynaud's phenomenon and/or other microvascular diseases and relative symptoms of poor circulation, such as pain. Such topical creams can also be useful in stimulating hemodynamic blood flow into tissue, including injured tissue. An increase in blood flow (or the maintenance of normal flow) into such tissue can ensure the presence of natural healing factors. This property can be of considerable benefit to patients who have incurred traumatic cuts, abrasions and/or surgical incisions. For such uses, antibiotic and/or topical anesthetics can be incorporated into selected formulations.

In one aspect, the invention related to water-based topical creams comprising nitroglycerin; one or more penetration enhancers; at least about 60% of an aqueous solvent by weight of the cream; and one or more pH-adjusters in an amount sufficient to provide pH of the cream at from about 4 to about 6.

In a further aspect, the invention relates to water-based topical creams comprising: nitroglycerin; and at least about 60% of an aqueous solvent by weight of the cream, wherein the cream has a six-month accelerated decomposition measurement of at least about 60% nitroglycerin.

It is contemplated that the disclosed compositions can be used in connection with the disclosed methods of making, the disclosed methods of using, and/or the disclosed kits.

Pharmaceutical compositions of the disclosed invention can be provided in a form suitable for topical use such as, for example, an aerosol, cream, ointment, lotion, gels and the like. Further, the compositions can be in a form suitable for use in transdermal devices. These formulations can be prepared via conventional processing methods.

1. Ingredients

The disclosed topical creams comprise various ingredients. It is contemplated that one or more disclosed ingredients can be included in the disclosed creams. It is also contemplated that one or more disclosed ingredients can be omitted from the disclosed creams. It is understood that various equivalents to the disclosed ingredients are known to those of skill in the art and can be substituted for the disclosed ingredients.

a. Nitroglycerin

Nitroglycerin (NG or NTG), also known as glyceryl trinitrate, trinitroglycerin (TNG) trinitroglycerine, 1,2,3-trinitropropane and glyceryl trinitrate, is a heavy, colorless, oily, explosive liquid obtained by nitrating glycerol. Nitroglycerin can be used medically as a vasodilator to treat heart conditions, such as angina and chronic heart failure.

In one aspect, nitroglycerin itself is a nitrovasodilator, which is denitrated to produce the active metabolite NO. Without wishing to be bound by theory, it is believed that clinically relevant denitration of nitroglycerin to produce 1,2-glyceryl dinitrate (GDN) and NO is catalysed by mitochondrial aldehyde dehydrogenase (mtALDH). NO is a potent activator of guanylyl cyclase (GC) by heme-dependent mechanisms; this activation results in cGMP formation from guanosine triphosphate (GTP). Thus, NO increases the level of cGMP within the cell.

Nitroglycerin can be obtained commercially as a 10% solution in propylene glycol. The disclosed compositions can be prepared directly from the commercially available 10% solution. It is understood that nitroglycerin can be light sensitive. It is also understood that nitroglycerin can be reactive with certain plastics. In one aspect, the disclosed nitroglycerin compositions can be stored in containers that prevent exposure to light and are either epoxy-coated or polypropylene/polyethylene lined.

In one aspect, the disclosed creams can comprise an amount of nitroglycerin. For example, in various aspects, a cream can comprise from about 0.1% to about 3%, from about 0.1% to about 1%, from about 0.1% to about 2%, from about 0.5% to about 3%, from about 0.5% to about 1%, from about 0.5% to about 2%, from about 1% to about 3%, from about 1% to about 2%, from about 0.2% to about 8%, or from about 0.3% to about 9% nitroglycerin by weight of the cream. In a further aspect, a cream can comprise an effective amount of nitroglycerin. In a yet further aspect, a cream can comprise a therapeutically effective amount of nitroglycerin. In a still further aspect, a cream can comprise an amount of nitroglycerin sufficiently low as to avoid unwanted side-effects.

b. Penetration Enhancers

A penetration enhancer is an agent known to accelerate the delivery of the drug through the skin. These agents also have been referred to as accelerants, adjuvants, and absorption promoters, and are collectively referred to herein as “enhancers.” This class of agents includes those with diverse mechanisms of action including those which have the function of improving the solubility and diffusibility of the drug, and those which improve percutaneous absorption by changing the ability of the stratum corneum to retain moisture, softening the skin, improving the skin's permeability, acting as penetration assistants or hair-follicle openers or changing the state of the skin such as the boundary layer.

Suitable enhancers that can be employed with the disclosed compositions include isopropyl palmitate, isopropyl myristate, laurocapram, and mixtures thereof. Suitable penetration enhancers include biocompatible, non-toxic organic esters in which assists as a solubilizing vehicle for carrying cosmetic or pharmaceutically active compounds across the skin of a mammal. One or more penetration enhancers can be employed in the disclosed compositions in an effective amount.

Useful esters include fatty mono esters having a structure obtainable by replacing the active hydrogen of a fatty acid having 4 to 22 carbon atoms (e.g., 8 to 18 carbon atoms or 12 carbon atoms) by the alkyl group of a monohydric alcohol. The fatty acid can be saturated or unsaturated and more typically is saturated. The monohydric alcohol typically contains 2 to 8 carbon atoms and more typically 2 to 5 carbon atoms, a particular example being 3 carbon atoms. Acceptable esters for this purpose include isopropyl esters. Preferably, the ester is isopropyl myristate or isopropyl palmitate, with isopropyl palmitate being particularly preferred.

Polar lipids such as CIS-unsaturated fatty acids can be effective enhancers for lipophilic or intermediate polarity drug molecules. They are less destructive to the skin than aggressive solvents such as dimethyl sulfoxide.

In one aspect, the disclosed creams can comprise an amount of one or more penetration enhancers. For example, in various aspects, a cream can comprise from about 0.1% to about 10%, from about 0.5% to about 10%, from about 1% to about 10%, from about 2% to about 10%, from about 1% to about 5%, from about 2% to about 5%, from about 1.5% to about 2.5%, from about 1% to about 10%, from about 5% to about 10%, from about 1% to about 4%, from about 1% to about 3%, from about 1% to about 2%, or from about 2% to about 3% of one or more penetration enhancers by weight of the cream. In a further aspect, a cream can comprise an effective amount of one or more penetration enhancers. In a yet further aspect, a cream can comprise a therapeutically effective amount of one or more penetration enhancers. In a still further aspect, a cream can comprise an amount of one or more penetration enhancers sufficiently low as to avoid unwanted side-effects.

c. Aqueous Solvent

The disclosed compositions typically include at least about 60% of an aqueous solvent by weight of the cream. As used herein, an aqueous solvent refers to a liquid or combination of liquids that mix uniformly with water. For example, the aqueous solvent typically comprises water or is at least miscible with water. In further aspects, a disclosed cream can comprise at least about 65%, at least about 70%, at least about 75%, or at least about 80% of an aqueous solvent by weight of the cream. For example, the aqueous solvent can be present as from about 60% to about 95%, from about 60% to about 90%, from about 75% to about 90%, or from about 80% to about 85% by weight of the cream.

In one aspect, the aqueous solvent comprises water. In further aspects, the aqueous solvent comprises a mixture of water and one or more alcohols, for example, ethanol. In yet further aspects, the one or more alcohols can be selected from methanol, ethanol, isopropanol, glycerol, propylene glycol, and mixtures thereof. An alcohol water mixture can be provided in a ratio of, for example, about 95:5 alcohol(s):water, about 90:10 alcohol(s):water, about 85:15 alcohol(s):water, about 80:20 alcohol(s):water, about 75:25 alcohol(s):water, about 70:30 alcohol(s):water, about 65:35 alcohol(s):water, about 60:40 alcohol(s):water, about 55:45 alcohol(s):water, about 50:50 alcohol(s):water, about 45:55 alcohol(s):water, about 40:60 alcohol(s):water, about 35:65 alcohol(s):water, about 30:70 alcohol(s):water, about 25:75 alcohol(s):water, about 20:80 alcohol(s):water, about 15:85 alcohol(s):water, about 10:90 alcohol(s):water, or about 5:95 alcohol(s):water.

Suitable biocompatible organic dihydric and polyhydric alcohol solvents may be any non-toxic di or polyalcohol in which the polar lipid and the active compound are soluble, and which assists as a solubilizing vehicle for carrying active compounds across the skin of a mammal. Acceptable dihydric and polyalcohols for this purpose include, but are not limited to di- and tri-alcohol alkanes. Typically the alcohols contain 3 to 8 carbon atoms and more typically 3 to 5 carbon atoms and are saturated alcohols. Preferably, the polyalcohol is propylene glycol or glycerol, with propylene glycol being particularly preferred.

In one aspect, the aqueous solvent can comprise one or more alcohols in the substantial absence of water. Without wishing to be bound by theory, when the disclosed creams comprise about 85% of an aqueous solvent prepared with ethanol alone, the cream can be formed, albeit having a gel-like consistency, while preparations with the likes of propylene glycol or glycerol typically yield a thicker cream.

In still further aspects, the one or more alcohols can be selected from polyalkyelene oxides, including polyethylene oxide, polypropylene oxide, and mixtures thereof. It is contemplated that polymeric co-solvents can be selected having molecular weights suitable for the formation of a cream (e.g., low molecular weights) as part of the aqueous solvent.

d. Thickeners

In one aspect, the disclosed creams further comprise at least one thickener. Suitable thickeners include anionic polymers such as polyacrylic acid (CARBOPOL® by B.F. Goodrich Specialty Polymers and Chemicals Division of Cleveland, Ohio), carboxymethylcellulose, and the like. Preferred thickeners employed in the present invention include methylcellulose, polyethylene glycol, and acrylic acid polymers. Carbopol 934P and Carbopol 940, commercially available from B.F. Goodrich Co., when neutralized, are suitable acrylic acid polymers. A preferred polyethylene glycol is polyethylene glycol 8000. A preferred methylcellulose is methylcellulose 4000. Additional thickeners, enhancers and adjuvants may generally be found in United States Pharmacopeia/National Formulary (2000); Remington's The Science and Practice of Pharmacy, Meade Publishing Co.

In one aspect, the disclosed creams can comprise an amount of one or more thickeners. In various aspects, thickener(s) can be present in an amount of about 0. 1 to about 5%, for example from about 0.25% to about 4%, from about 0.5% to about 3%, from about 0.5% to about 2%, from about 0.5% to about 1%, from about 1% to about 2%, or about 1.6% by weight. In a further aspect, a cream can comprise an effective amount of one or more thickeners. In a still further aspect, a cream can comprise an amount of one or more thickeners sufficiently low as to avoid unwanted side-effects.

One exemplary class of thickeners that can be employed in connection with the disclosed invention is Carbomers. Carbomers are highly ionic, acidic, white, fluffy powders with a slight characteristic odor. See “Final Report on the Safety Assessment of Carbomers-934,-910,-934P, -940,-941, and -962,” Journal of the American College of Toxicology, 1 (2), 1982. Carbomers are used as thickening, suspending, dispersing, and emulsifying agents. They are widely used to provide emulsion stabilization and rheologic control. Carbomer dispersions typically show increased viscosity with increasing concentration of polymer.

Carbomer polymers are used in pharmaceutical products as thickening, dispersing, and emulsifying agents. They are also used to control the release of medicaments from time-release tablets or from entrapped systems. In cosmetic preparations, they are frequently used in their neutralized form—that is, as a gel. The Carbomers are normally used in cosmetics between a pH of 6.0 and 9.0. Clinical studies with Carbomer-934 and its various salts showed that these polymers have low potential for skin irritation and sensitization at concentrations of 0.5%, 5%, 10%, and 100%.

Carbomers are largely insoluble in water and in the majority of common solvents. When neutralized (with bases, e.g., hydroxides or amines), Carbomers can be soluble in water, alcohol and glycerin. Carbomers are hygroscopic in nature, swelling to many times their original volume when in contact with a solvent. Such swollen particles remain discrete in various mucilaginous or colloidal dispersions. Although swelling is inherently caused by their hydrophilic nature, “maximum volume swell” does not typically occur in water until the polymers are converted to partial organic or inorganic salts. The increased volume is generally stable at all pH levels, but increases as neutralization increases. Maximum volume occurs at 50-90% neutralization, with a neutralization of 75% normally occurring at pH 7.0.

The finely divided, free-flowing Carbomer powders readily disperse in water to yield a low viscosity acid solution. When neutralized, the solution is transformed into a clear, stable gel. In acidic aqueous media (pH 3.5-4.0), Carbomers yield dispersions of low to moderate viscosity. Between pH 5.0 and 10.0, the polymers reach their optimal viscosity when they set into an emollient gel. At pH levels above 10, the gel structure collapses and viscosity drops.

Carbomers are generally used in a concentration of up to 50%. However, in cosmetics, Carbomers are normally used at concentrations below about 1%. When Carbomers are used in unneutralized form, however, their concentration can be as high as about 2%.

e. Emulsifiers

In one aspect, the disclosed creams further comprise at least one emulsifier. The emulsifier can be a non-ionic surface active agent. Suitable non-ionic surfactants include the polysorbates, which are mixtures of partial esters of sorbitol and its mono- and dianhydrides, typically condensed with approximately 20 mol of ethylene oxide; polyethyoxylated alkyl ethers and esters, in which the alkyl chain can be either saturated, unsaturated, branched or linear; polyethoxylated alkyl phenols, in which the hydrophobic group normally octyl or nonylphenol; and poloxamers, polyoxyethylene-polyoxypropylene block copolmyers, in which the polyoxypropylene chain acts as the hydrophobic moiety.

Some commercially available non-ionic surfactants are Brij 98, Brij 78, polyoxyl 40 stearate, and polysorbate 80. Brij 98 and Brij 78 are polyethylene glycol fatty alcohol ethers. Polyoxyl 40 stearate is a mixture of mono and distearate esters of polyoxyethylene and of free polyoxyethylene. Polysorbate 80 is polyoxyethylene (20) sorbitan mono-oleate, which is commercially available under the trade name TWEEN80®.

In one aspect, the disclosed creams can comprise an amount of one or more emulsifiers. An emulsifier can be present in an amount of, for example, from about 0.1% to about 2%, from about 0.2% to about 1%, from about 0.4% to about 2%, or from about 0.2% to about 2% by weight of the cream. In a further aspect, a cream can comprise an effective amount of one or more emulsifiers. In a still further aspect, a cream can comprise an amount of one or more emulsifiers sufficiently low as to avoid unwanted side-effects.

It is understood that the disclosed creams can comprise at least one thickener and/or at least one emulsifier.

f. pH-Adjusters

In one aspect, the disclosed water-based topical creams comprise one or more pH-adjusters in an amount sufficient to provide pH of the cream at from about 4 to about 6. For example, one or more pH-adjusters can be present in an amount of from about 0.01% to about 1.0%, for example, from about 0.05% to about 0.9%, from about 0.1% to about 0.8%, or from about 0.1% to about 0.7% by weight of the cream.

The pH-adjuster(s) are typically Bronsted-Lowry and/or Lewis acids or bases. In one aspect, a pH-adjuster is a Bronsted-Lowry and/or Lewis base. In a further aspect, the one or more pH-adjusters can be a base selected from hydroxides, carbonates, ammonia, amines, borates, phosphates, and citrates. For example, a pH-adjuster can be an amine selected from triethylamine, diethylmethylamine, ethyldimethylamine, triethanolamine, and isopropyldimethylamine.

One of skill in the art can readily understand that a pH-adjuster can be included in the disclosed compositions with reference to a pH-endpoint, rather than with reference to a particular amount. For example, an amount of a pH-adjuster can be added to a composition and the pH can be measured. If desired, additional amounts of pH-adjuster can then be added to the composition until the pH measurement is within the desired pH range.

g. Auxiliary Ingredients

The pharmaceutical formulations described herein can further include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Thus, the disclosed compositions can optionally contain auxiliary ingredients, such as flavorings, fragrances, preservatives and/or coloring agents. In one aspect, a cream can comprise one or more preservatives, for example, antifungals, antimicrobials and/or antioxidants. Suitable preservatives include methylparabens, propylparabens, chrorhexidine digluconate, and butylhydroxy toluene (BHT). Suitable auxiliary components also include a topical anesthetic such as lidocaine and dibucaine, over-the-counter pain relievers such as Ibuprofen or Naproxen, or an appropriate antibiotic such as tetracycline.

In one aspect, the disclosed creams can comprise an amount of one or more auxiliary components. It is understood that the various one or more auxiliary components can be provided in independent amounts.

For example, in various aspects, a cream can comprise from about 0.01% to about 15%, from about 0.05% to about 15%, from about 0.1% to about 15%, from about 0.5% to about 15%, from about 1% to about 15%, from about 2% to about 15%, from about 3% to about 15%, or from about 5% to about 15% of an auxiliary component by weight of the cream. As further examples, a cream can comprise from about 0.01% to about 10%, from about 0.1% to about 10%, from about 1% to about 10%, from about 1% to about 5%, from about 1% to about 3%, from about 1% to about 2%, from about 0.01% to about 1%, from about 0.05% to about 1%, from about 0.1% to about 1%, from about 0.5% to about 1%, from about 0.01% to about 2%, from about 0.05% to about 2%, from about 0.1% to about 2%, from about 0.5% to about 2%, from about 0.01% to about 3%, from about 0.05% to about 3%, from about 0.1% to about 3%, or from about 0.5% to about 3% of an auxiliary component by weight of the cream. In a further aspect, a cream can comprise an effective amount of an auxiliary component. In a yet further aspect, a cream can comprise a therapeutically effective amount of an auxiliary component. In a still further aspect, a cream can comprise an amount of an auxiliary component sufficiently low as to avoid unwanted side-effects.

In still further aspects, the creams can further comprise other liquid components, for example, fatty alcohols. Suitable fatty alcohols include capryl alcohol (1-octanol), 2-ethyl hexanoyl, pelargonic alcohol (1-nonanol), capric alcohol (1-decanol, decyl alcohol), 1-dodecanol (lauryl alcohol), myristyl alcohol (1-tetradecanol), cetyl alcohol (1-hexadecanol), palmitoleyl alcohol (cis-9-hexadecen-1-ol), stearyl alcohol (1-octadecanol), isostearyl alcohol (16-methylheptadecan-1-ol), elaidyl alcohol (9E-octadecen-1-ol), oleyl alcohol (cis-9-octadecen-1-ol), linoleyl alcohol (9Z, 12Z-octadecadien-1-ol), elaidolinoleyl alcohol (9E, 12E-octadecadien-1-ol), linolenyl alcohol (9Z, 12Z, 15Z-octadecatrien-1-ol), elaidolinolenyl alcohol (9E, 12E, 15-E-octadecatrien-1-ol), ricinoleyl alcohol (1²-hydroxy-9-octadecen-1-ol), arachidyl alcohol (1-eicosanol), behenyl alcohol (1-docosanol), erucyl alcohol (cis-13-docosen-1-ol), lignoceryl alcohol (1-tetracosanol), ceryl alcohol (1-hexacosanol), montanyl alcohol, cluytyl alcohol (1-octacosanol), myricyl alcohol, melissyl alcohol (1-triacontanol), and geddyl alcohol (1-tetratriacontanol). When water/fatty alcohol mixtures are employed, the mixture comprises an emulsion. Typically, when present, fatty alcohols are employed in low amounts, for example, from about 0.1 to about 2%, from about 0.5 to about 2%, from about 0.1 to about 1%, or from about 0.5 to about 1% by weight of the total composition.

In still further aspects, the aqueous solvent can further comprise other liquid components, for example mineral oil (e.g., light mineral oil (also referred to as liquid petrolatum or baby oil), cottonseed oil, castor oil and the like) can be used as a component of the aqueous solvent. When water/oil mixtures are employed, the mixture comprises an emulsion. Thus, in still further aspects, the cream can, for example, comprise about 85% of an aqueous solvent by weight of the cream, the aqueous solvent comprising about 55% water and about 30% oil.

2. Properties

The disclosed topical creams exhibit various properties. It is understood that viscosity and pH can be related to cream performance. It is also understood that viscosity and pH can be related to cream stability.

a. PH

In one aspect, the pH of the disclosed creams is less than about 6, less than about 5.5, or from about 5 to about 5.3. In a further aspect, the pH of the disclosed creams is from about 4 to about 6, for example, from about 4.5 to about 6, from about 4.5 to about 5.5, from about 4.5 to about 5, from about 5 to about 6, from about 5 to about 5.5, from about 5.5 to about 6, from about 5.1 to about 5.3, from about 5 to about 5.2, or from about 5.2 to about 5.4. As disclosed herein, the pH value can be adjusted by adding a pH-adjuster and/or a suitable buffer system, such as phosphate, borate or citrate based buffers.

Without wishing to be bound by theory, it is believed that a relationship exists between cream pH and stability of nitroglycerin in the cream. Without wishing to be bound by theory, it also is believed that a relationship exists between cream pH and integrity of the cream (as observed by viscosity and/or droplet formation).

b. Viscosity

Viscosity refers to a measure of the resistance of a fluid to being deformed by either shear stress or extensional stress. It is commonly perceived as “thickness,” or resistance to flow. With reference to a water-based cream, viscosity can describe the tendency of the cream to retain a semi-solid state, as compared to a more liquid-like state. In general, a low viscosity can indicate that a particular composition is less like a cream and more like a liquid.

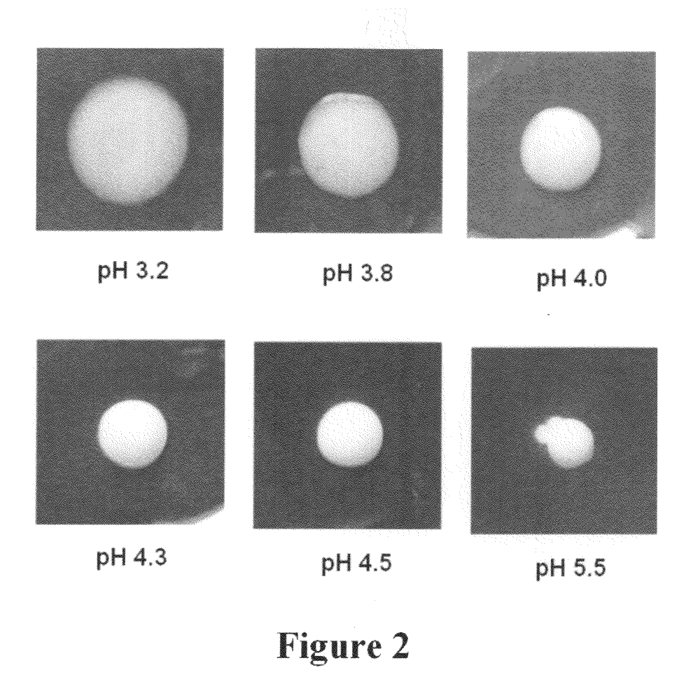

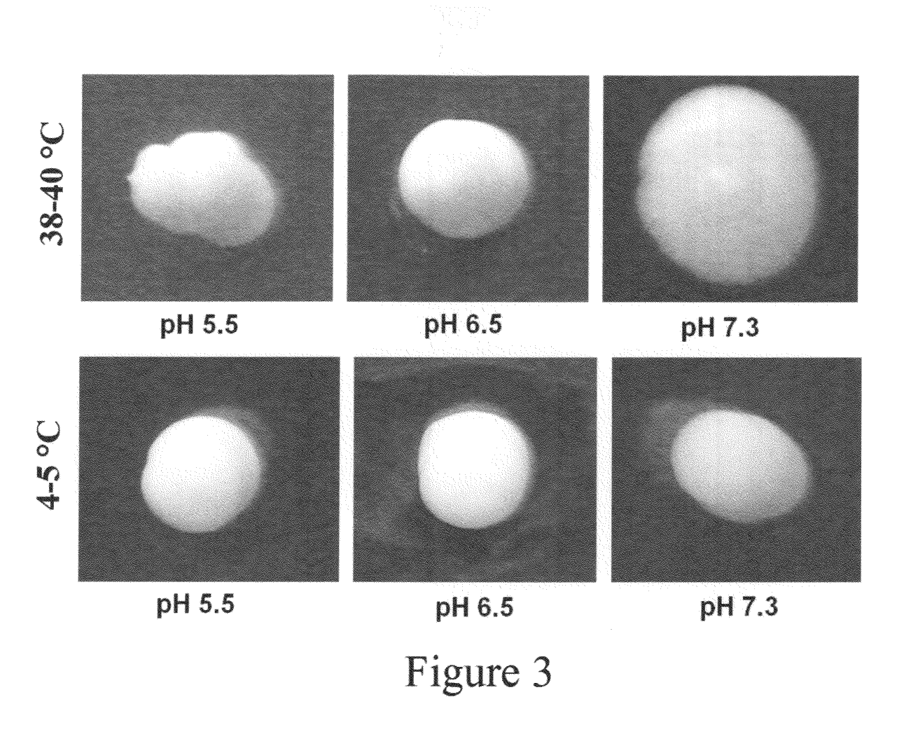

Viscosity of the disclosed compositions can be characterized by a viscometer, as described herein or can be evaluated by function of droplet diameter, which is correlated to composition viscosity, as shown in FIG. 2 and FIG. 3.

TABLE 1
DROPLET DIAMETER AS A FUNCTION OF PH

| pH | Diameter (cm) |
|---|---|
| 3.2 (no base added) | 4.5 |
| 3.8 | 3.7 |
| 4.0 | 2.9 |
| 4.3 | 2.0 |
| 4.5 | 1.8 |
| 5.5 | 1.2 |

Table 1 presents data from the experiment shown in FIG. 2. These data indicate that droplet diameter generally increases for a cream formulation as pH is decreased, which correlates with decreased viscosity of the cream at a lower pH. Without wishing to be bound by theory, it is believed that decreased pH can decrease the gel character of the cream.

TABLE 2
VISCOSITY STABILITY AS A FUNCTION OF PH

Diameter (cm)

| Initial pH | 4-5° C. | 38-40° C. |
|---|---|---|
| 5.5 | 1.3 | 1.3 |
| 6.5 | 1.3 | 1.6 |
| 7.3 | 1.3 | 2.5 |

Table 2 presents data from the experiment shown in FIG. 3. Creams contained 0.6% nitroglycerin and were incubated for five months. These data indicate that droplet diameter is generally constant when varying intial pH creams and incubated at a temperature of 4-5° C. (both relatively high and relatively low pH creams), while droplet increases for creams of relatively higher initial pH as the temperature is increased to 38-40° C. over time.

c. Stability

In a further aspect, the disclosed creams are stable, as compared with conventional water-based creams. For example, a water-based topical cream can comprise nitroglycerin; and at least about 60% of an aqueous solvent by weight of the cream, wherein the cream has a six-month accelerated decomposition measurement of at least about 60% nitroglycerin. In further aspects, the six-month accelerated decomposition measurement can be at least about 65%, at least about 70%, at least about 75%, at least about 80%, at least about 85%, or at least about 90%.

Again, without wishing to be bound by theory, it is believed that a relationship exists between cream pH and stability of nitroglycerin in the cream. In general, a cream exhibiting a pH of from about 4 to about 6, for example from about 5 to about 5.5 or from about 5 to about 5.3, can be more stable than conventional water-based creams. In one aspect, a disclosed cream can have enhanced nitroglycerin stability as well as enhanced viscosity stability of cream base.

3. Example Formulations

In one aspect, a disclosed cream can comprise from about 0.01% to about 3% by weight of nitroglycerin; from about 1% to about 10% by weight of a penetration enhancer; from about 60% to about 95% by weight of an aqueous solvent; from about 0.5 to about 3% by weight of one or more thickeners; about 0. 1 to about 2% by weight of an emulsifier; and from about 0.1% to about 0.7% by weight of an amine base.

In one aspect, a disclosed cream can comprise from about 0.01% to about 3% by weight of nitroglycerin; from about 1% to about 3% by weight of isopropyl palmitate; from about 60% to about 90% by weight of aqueous solvent; from about 0.5% to about 1% by weight of a crosslinked acrylic acid-based polymer; from about 0.2% to about 0.8% by weight of methyl cellulose; about 0.2% to about 1% by weight of polyethylene glycol; about 0.2% to about 1% by weight of Brij or Tween 80; and from about 0.1% to about 0.6% by weight of triethanolamine, wherein the cream has a pH of from about 5 to about 5.5.

In one aspect, a disclosed cream can comprise from about 0.3 to about 2% by weight of nitroglycerin; from about 1% to about 3% by weight of isopropyl palmitate; from about 70% to about 90% by weight of aqueous solvent; from about 0.5 to about 1% by weight of a crosslinked acrylic acid-based polymer; from about 0.2 to about 0.8% by weight of methyl cellulose; from about 0.2% to about 1% polyethylene glycol; from about 0.2% to about 1% by weight of Brij or polysorbate 80; and from about 0.1% to about 0.6% by weight of triethanolamine, wherein the cream has a pH of from about 5 to about 5.5.

Some exemplary formulations are shown in Table 3, below:

TABLE 3
EXEMPLARY FORMULATIONS

| | 1* | 2* | 3* | 4** | 5 | 6*** | 7**** | 8**** |
|---|---|---|---|---|---|---|---|---|
| Water | 83.50% | 70.65% | 85.35% | 57.06% | 55.20% | 55.0% | 77.20% | 75.44% |
| Alcohol | — | — | — | 27.2% | — | — | — | — |
| Oil | — | — | — | — | — | 30.0% | — | — |
| 10% NTG | 12.0% | 25.0% | 6.0% | 6.0% | 6.0% | 6.0% | 6.0% | 10.0% |
| PG | — | — | 4.0% | 5.0% | 32.0% | 4.0% | 8.0% | 5.0% |
| IPP | 2.0% | 2.0% | 2.0% | 2.0% | 2.0% | 2.0% | 1.8% | 2.0% |
| Carb | 0.7% | 0.7% | 0.7% | 0.8% | 1.2% | 0.8% | 0.7% | 0.7% |
| MC | 0.4% | 0.4% | 0.5% | 0.5% | 0.5% | 0.5% | 0.4% | 0.4% |
| PEG8000 | 0.5% | 0.5% | 0.5% | 0.5% | 0.5% | 0.5% | — | 0.5% |
| Brij | — | — | — | 0.5% | 0.5% | 0.5% | 0.4% | — |
| Polysorbate | 0.5% | 0.5% | 0.4% | — | — | — | — | 0.5% |
| 80 | | | | | | | | |
| TEA | 0.3% | 0.15% | 0.35% | 0.34% | 0.2% | 0.6% | 0.36% | 0.36% |
| MP | 0.08% | 0.08% | 0.08% | 0.08% | 0.08% | 0.08% | 0.07% | 0.08% |
| PP | 0.02% | 0.02% | 0.02% | 0.02% | 0.02% | 0.02% | 0.02% | 0.02% |
| BHT | — | — | — | — | — | — | 0.05% | — |
| Ibuprofen | — | — | — | — | — | — | 5.0% | — |
| Naproxen | — | — | — | — | — | — | — | 5.0% |

*Can also use other emulsifiers; can also use polyoxyl 40 stearate.
**Ethanol was used as alcohol in this example; can also use other alcohols (e.g., methanol or isopropanol)
***Light mineral oil was used as Oil in this example; can also use cottonseed oil, castor oil, etc.
****Ibuprofen and Naproxen: non-steroidal anti-inflamatory drugs (NSAID).

In Table 3, the following abbreviations were used: NTG=nitroglycerin, PG=propylene glycol, IPP=isopropyl palmitate, Carb.=carbopol 934P, MC=methyl cellulose, MP=methyl paraben, Brij=Brij 98, TEA=triethanolamine, PP=propyl paraben, PEG8000=polyethylene glycol 8000, BHT=butylated hydroxyltoluene. TEA can be used undiluted, or diluted as an aqueous solution. Nitroglycerin is commercially available as a 10% solution in propylene glycol. Example 1 in this Table is a 1.2% concentration of NTG, example 2 is a 2.5% concentration of NTG, and examples 3-7 are 0.6% concentration of NTG.

### C. Methods of Making

In one aspect, the disclosed invention relates to methods for preparing water-based topical creams, the method comprising the steps of: providing a mixture of aqueous solvent, one or more penetration enhancers, and, optionally, one or more preservatives; adding to the mixture one or more thickeners; adding to the mixture one or more emulsifiers; heating the mixture to at least a temperature sufficient to solubilize or melt the one or more thickeners and/or one or more emulsifiers; and adjusting the pH of the mixture to from about 4 to about 6 with a pH-adjuster. In a further aspect, the pH is adjusted before or during one or more of the providing, adding, and heating steps. In a yet further aspect, the pH is adjusted after the providing, adding, and heating steps. In one aspect, the cream comprises at least about 60% aqueous solvent by weight.

Typically, the mixture is provided as a uniform suspension (dispersion) of ingredients. In certain aspects, the mixture can appear as an emulsion or a solution.

In one aspect, the methods are single-vessel methods; that is, the steps can be performed in a single vessel. In contrast to conventional methods for preparing water-based creams containing nitroglycerin, multiple vessels are not required.

The sequence of addition can affect the integrity of the cream. For example, addition of an aqueous solvent (e.g., water or water/alcohol mixtures) to a mixture of penetration enhancer(s) (e.g., isopropyl palmitate), auxiliary ingredients (e.g., preservatives such as methyl paraben and propyl paraben), followed by addition of thickeners (e.g., Carbopol 934P, methyl cellulose, and/or polyethylene glycol) and/or emulsifiers (e.g. Brij 98 and/or TWEEN80), can provide a more consistent, uniform cream than other preparation methods.

In certain aspects, it can be desirable to heat the mixture or to heat an ingredient to be added to the mixture. Such an increase in temperature can melt the ingredient and/or can aid solubility/miscibility of the ingredient with the mixture. For example, the methods can further comprise a step of heating to a temperature sufficient to melt and/or solubilize a thickener (e.g., a Carbopol) and/or a step of heating to a temperature sufficient to melt and/or solubilize an emulsifier (e.g., Brij 98 or TWEEN80). Typically, the heating step facilitates uniform distribution of the thickener(s) and/or emulsifier(s) into the mixture. Accordingly, the heating step typically employs a temperature of at least about the melting temperature of any solid or semi-solid ingredients. In one aspect, the temperature sufficient to melt and/or solubilize is at least about room temperature, at least about 35° C., at least about 40° C., at least about 45° C., at least about 50° C., at least about 55° C., at least about 60° C., at least about 65° C., at least about 70° C., at least about 75° C., or at least about 80° C.

In various aspects, the heating step can be performed before the one or more emulsifiers are added and/or before the one or more thickeners are added. In further aspects, the heating step and the adding one or more thickeners and/or emulsifiers step can be performed substantially simultaneously. In further aspects, the heating step and the adding one or more thickeners and/or emulsifiers step can be performed substantially simultaneously.

The methods can further comprise the step of adjusting the pH of the mixture to from about 4 to about 6 with a pH-adjuster. The pH can be, for example, any pH from about 4 to about 6, from about 4.5 to about 6, from about about 5 to about 5.5, or from about 5 to about 5.3. The pH adjuster can be any pH adjuster disclosed herein, for example, a base selected from hydroxides, carbonates, ammonia, amines, borates, phosphates, and citrates or an amine selected from triethylamine, diethylmethylamine, ethyldimethylamine, triethanolamine, and isopropyldimethylamine.

The methods can further comprise the step of adding a pharmaceutically active ingredient. The pharmaceutically active ingredient can be introduced before, during, or after pH adjustment. In one aspect, the pharmaceutically active ingredient comprises nitroglycerin. Nitroglycerin can be employed in the disclosed methods as a 10% solution in propylene glycol. It is contemplated that nitroglycerin can alternatively be added in a solid form to the various formulations.

In further aspects, the pharmaceutically active ingredient can be selected from molecules, groups of molecules, complexes or substances administered to an organism for diagnostic, therapeutic, or preventative medical or veterinary purposes. This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like.

Additional examples of suitable pharmaceutically active ingredients that can be used in connection with the disclosed creams include radiosensitizers, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. Typically, the pharmaceutically active ingredients are selected for efficacy in topical administration and for compatibility with the cream.

It is contemplated that the disclosed methods of making can be used in connection with the disclosed compositions, the disclosed methods of using, and/or the disclosed kits.

### D. Methods of Using

Also disclosed are various methods of using the disclosed water-based creams. For example, the disclosed creams can be used in methods for treating circulatory disorders in co-adminstration methods for drug-induced insufficient circulation, in co-adminstration methods for disease-induced insufficient circulation, in co-adminstration methods for enhancing localized activity of systemically adminstered drug, in methods for enhancing circulation, and in methods for promoting wound healing.

In one aspect, the preferred amount of the disclosed cream used for is from about 0.2 to about 2 grams of the composition. For example, an amount of from about 0.25 to about 2 grams, from about 0.5 to about 2 grams, from about 0.75 to about 2 grams, from about 1 gram to about 2 grams, from about 0.2 to about 1 gram, from about 0.25 to about 1 gram, from about 0.5 to about 1 gram, from about 0.75 to about 1 gram, or from about 0.5 to about 1.5 grams. The amount to be administered relates to the concentration of active ingredient, the condition treated and/or the outcome desired, as well as to the area of a subject's skin to be treated. For example, in treating a small wound, a higher concentration of nitroglycerin and a smaller amount of cream (corresponding to a smaller surface area of skin to be treated) can be preferred. As another example, in treating peripheral neuropathy, a low to average concentration of nitroglycerin and a larger amount of cream (corresponding to a larger surface area of skin to be treated) can be preferred.

It is also understood that the amount administered can be determined by administering an amount to a subject, followed by observing the subject after administration for increased vasodilation, stimulated hemodynamic blood flow into injured tissue, treatment of a condition related to decreased blood flow, or other symptom. One of skill (e.g., a physician) can readily adjust the amount administered (i.e., dosage) after observing.

It is also understood that the disclosed creams can be employed to treat various disorders (e.g., circulatory disorders) as well as symptoms associated with the disorders (e.g., pain associated with poor circulation).

It is contemplated that the disclosed methods of using can be used in connection with the disclosed methods of making, the disclosed compositions, and/or the disclosed kits.

1. Methods for Treating Circulatory Disorder

In one aspect, the invention relates to methods of treating a circulatory disorder comprising topically administering to a mammal a therapeutically effective amount of water-based topical cream comprising nitroglycerin and at least about 60% by weight of an aqueous solvent and having a pH of from about 4 to about 6, thereby treating the disorder in the mammal. It is understood that the creams employed in these methods can be modified and adapted as disclosed herein.

In a further aspect, the mammal is human. In various aspects, the mammal can be diagnosed with the circulatory disorder prior to administration. That is, the disclosed methods can include identifying a mammal in need of treatment of the circulatory disorder.

Diseases that can be treated by topical administration of the disclosed creams include one or more of peripheral vascular disease, peripheral artery disease, male impotence, Raynaud's disease, diabetic peripheral neuropathy, vulvodynia, female anorgasmia, anal fissues, coronary artery disease, nocturnal leg cramps, restless leg syndrome, and wounds (for example, pressure wounds, heat burns, and chemical burns).

2. Co-Adminstration Methods for Drug-Induced Insufficient Circulation

In one aspect, the invention relates to methods of preventing or alleviating insufficient circulation comprising co-administering to a mammal a therapeutically effective amount of water-based topical cream comprising nitroglycerin and at least about 60% of an aqueous solvent by weight of the cream and having a pH of from about 4 to about 6 with a drug having a known side-effect of decreasing circulation, thereby preventing or alleviating the insufficient circulation in the mammal. It is understood that the creams employed in these methods can be modified and adapted as disclosed herein.

In a further aspect, the mammal is human. In various aspects, the mammal can be diagnosed with a need for the drug having a known side-effect of decreasing circulation prior to administration. That is, the disclosed methods can include identifying a mammal in need of a drug having a known side-effect of decreasing circulation.

The drug having a known side-effect of decreasing circulation can be administered by any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. The drug and the cream can be administered sequentially (e.g., drug before topical cream or drug after topical cream) or administered substantially simultaneously.

The drug can be any drug having a known side-effect of decreasing circulation. For example, the drug can be a chemotherapeutic. As a further example, the drug can be any drug having a side effect of vasoconstriction. In one aspect, the drug having a known side-effect of decreasing circulation is a tobacco product, for example, cigarettes, cigars, snuff, or other nicotine-containing agents.

3. Co-Administration Methods for Disease-Induced Insufficient Circulation

In one aspect, the invention relates to methods of preventing or alleviating insufficient circulation comprising co-administering to a mammal a therapeutically effective amount of water-based topical cream comprising nitroglycerin and at least about 60% of an aqueous solvent by weight of the cream and having a pH of from about 4 to about 6 with a drug known to treat a disorder associated with insufficient circulation, thereby preventing or alleviating the insufficient circulation in the mammal. It is understood that the creams employed in these methods can be modified and adapted as disclosed herein.

In a further aspect, the mammal is human. In various aspects, the mammal can be diagnosed with a disorder associated with insufficient circulation prior to administration. That is, the disclosed methods can include identifying a mammal in need of treatment of the disorder associated with insufficient circulation.

The disorder can be any disorder associated with insufficient circulation. For example, the disorder can be diabetes or Raynaud's disease

The drug known to treat a disorder associated with insufficient circulation can be administered by any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. The drug and the cream can be administered sequentially (e.g., drug before topical cream or drug after topical cream) or administered substantially simultaneously.

The drug can be any drug known to treat a disorder associated with insufficient circulation. For example, the drug can be insulin. As a further example, the drug can be any drug known to treat disorders involved in occlusion, e.g., peripheral artery disease and heart disease.

4. Co-Administration Methods for Enhancing Localized Activity of Systemically Adminstered Drug

In one aspect, the invention relates to methods of enhancing localized activity of a systemically administered drug comprising topically administering to an external surface area of a mammal a therapeutically effective amount of water-based topical cream comprising nitroglycerin and at least about 60% of an aqueous solvent by weight of the cream; and substantially simultaneously administering a drug having a desired systemic effect, thereby enhancing the desired systemic effect proximate to the external surface area of the mammal. It is understood that the creams employed in these methods can be modified and adapted as disclosed herein.

In a further aspect, the mammal is human. In various aspects, the mammal can be diagnosed with a need for localized activity of the drug having a desired systemic effect prior to administration. That is, the disclosed methods can include identifying a mammal in need of localized activity of the drug having a desired systemic effect.

The desired systemic effect can be any desired systemic effect that can be desired to be localized in an external surface area of a mammal. For example, the desired systemic effect can be pain relief or anti-inflammation.

The drug having a desired systemic effect can be administered by any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. The drug and the cream can be administered sequentially (e.g., drug before topical cream or drug after topical cream) or administered substantially simultaneously.

The drug can be any drug known to treat a disorder associated with insufficient circulation that can be desired to be localized in an external surface area of a mammal. For example, the drug can be a non-steroidal anti-inflammatory drug (NSAID) (e.g., ibuprofen or naproxen), or an analgesic. In a further example, the drug can be another drug used to increase circulation, e.g., papaverine, phentolamine, adenosine, and/or adrenaline.

5. Methods for Enhancing Circulation

In one aspect, the invention relates to methods of enhancing circulation comprising topically administering to an external surface area of a mammal an effective amount of water-based topical cream comprising nitroglycerin and at least about 60% of an aqueous solvent by weight of the cream, thereby increasing circulation in the vasculature proximate to the external surface area of the mammal to a level greater than that of the external surface area prior to administration. It is understood that the creams employed in these methods can be modified and adapted as disclosed herein.

In a further aspect, the mammal is human. In various aspects, the mammal can be diagnosed with a need for enhanced circulation prior to administration. That is, the disclosed methods can include identifying a mammal in need of enhanced circulation.

Typically, the circulation is increased by at least about 5%, for example, at least about 10%, at least about 20%, at least about 25%, at least about 33%, at least about 50%, at least about 66%, at least about 75%, at least about 100%, or at least about 200%. The circulation increase can be measured by conventional techniques.

In a further aspect, the method can further comprise the step of exfoliating the skin of the external surface area. That is, the surface of the skin can be prepared for receipt of the cream and/or other topically administered materials. In a yet further aspect, the method can comprise the step of topically administering to the external surface area an effective amount of one or more nutrients. The nutrients and the cream can be administered sequentially (e.g., nutrients before topical cream or nutrients after topical cream) or administered substantially simultaneously.

In various aspects, the nutrients can be one or more of vitamins, minerals, moisturizers, oils and butters, herbs and herb oils, botanicals, aloe vera, fatty acids, vegetable extracts such as carrot extract, coenzymes, and /or proteins.

In a further aspect, the method can prevent or treat nail disorders and/or improve brittle, peeling, soft nails.

In a further aspect, the method can increase blood flow to the scalp (administration of the creams alone or with nutrients), thereby promoting hair growth and prevention of hair loss.

6. Methods for Promoting Wound Healing

In one aspect, the invention relates to methods of promoting wound healing comprising topically administering at or proximate to injured tissue of a mammal an effective amount of water-based topical cream comprising nitroglycerin and at least about 60% of an aqueous solvent by weight of the cream, thereby increasing hemodynamic blood flow into the injured tissue. It is understood that the creams employed in these methods can be modified and adapted as disclosed herein.

In a further aspect, the mammal is human. In various aspects, the mammal can be diagnosed with a wound prior to administration. That is, the disclosed methods can include identifying a mammal in need of wound treatment.

Typically, the hemodynamic blood flow into the injured tissue is increased by at least about 5%, for example, at least about 10%, at least about 20%, at least about 25%, at least about 33%, at least about 50%, at least about 66%, at least about 75%, at least about 100%, at least about 150%, or at least about 200%. The hemodynamic blood flow increase can be measured by conventional techniques, for example Doppler or ultrasound techniques.

In various aspects, the the injury can be a bruise, a laceration, an abrasion, a burn, frostbite, a surgical incision, or a pressure wound.

In a further aspect, the invention relates to methods of preventing wound formation comprising topically administering at or proximate to tissue of a mammal an effective amount of water-based topical cream comprising nitroglycerin and at least about 60% of an aqueous solvent by weight of the cream, prior to an injury occuring, thereby increasing hemodynamic blood flow into the injured tissue. In one aspect, the method can prevent formation of pressure wounds (i.e., tissue necrosis caused by a patient lying on her back for a long time). A patient on her back in bed for a long time because of an illness or injury can be prophylactically treated with the disclosed creams to prevent pressure wounds, for example bed sores or wounds caused by a device.

7. Artery Dilation

The radial artery, which is located on the outer side of the forearm, can be used in interventional procedures, such as cardiac catheterization, to provide access to the arterial blood supply. In order to facilitate successful catheterization of the artery, a dilated artery free of arterial spasm is desirable. The disclosed creams can be administered into tissue proximate to an artery, thereby providing dilated artery free of arterial spasm, suitable for interventional procedures, such as catheterization. Radial artery diameter can be measured with ultrasound.

Thus, in a further aspect, the invention relates to methods of artery dilation for use in catheterization comprising topically administering at or proximate to an artery of a mammal an effective amount of water-based topical cream comprising nitroglycerin and at least about 60% of an aqueous solvent by weight of the cream, thereby promoting dilation of the artery, and catherizing the dilated artery. It is understood that the creams employed in these methods can be modified and adapted as disclosed herein. In one aspect, the artery is a radial artery. In a further aspect, catherization is arterial catherization.

### E. Kits

In various aspects, the invention also related to kits comprising the disclosed creams. For example, a kit can comprise a drug having a known side-effect of decreasing circulation and a water-based topical cream comprising nitroglycerin, a drug having a desired systemic effect and a water-based topical cream comprising nitroglycerin, a drug known to treat a disorder associated with insufficient circulation and a water-based topical cream comprising nitroglycerin, or a kit comprising one or more nutrients and a water-based topical cream comprising nitroglycerin. It is understood that the creams employed in these kits can be modified and adapted as disclosed herein. For example, in a further aspect, the cream further comprises at least about 60% of an aqueous solvent by weight of the cream and has a pH of from about 4 to about 6.

The kits can comprise creams co-packaged, co-formulated, and/or co-delivered with other components. For example, a drug manufacturer, a drug reseller, a physician, a compounding shop, or a pharmacist can provide a kit comprising a disclosed cream and another component for delivery to a patient.

In a further aspect, the invention relates to kits comprising a drug having a known side-effect of decreasing circulation and a water-based topical cream comprising nitroglycerin.

In a further aspect, the invention relates to kits comprising a drug having a desired systemic effect and a water-based topical cream comprising nitroglycerin.

In a further aspect, the invention relates to kits comprising a drug known to treat a disorder associated with insufficient circulation and a water-based topical cream comprising nitroglycerin.

In a further aspect, the invention relates to kits comprising one or more nutrients and a water-based topical cream comprising nitroglycerin.

It is contemplated that the disclosed kits can be used in connection with the disclosed methods of making, the disclosed methods of using, and/or the disclosed compositions.

### F. Experimental

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Accelerated Decomposition Measurements

Prepared creams are incubated at 4-5° C. and in a controlled 38-40° C. incubator with water to maintain a humid environment. At different incubation intervals starting at 0 time to 6 months, tubes are taken out from both 4-5° C. and 38-40° C. and each sample is analyzed at room temperature for pH, viscosity, and nitroglycerin content by HPLC.

a. HPLC Measurements

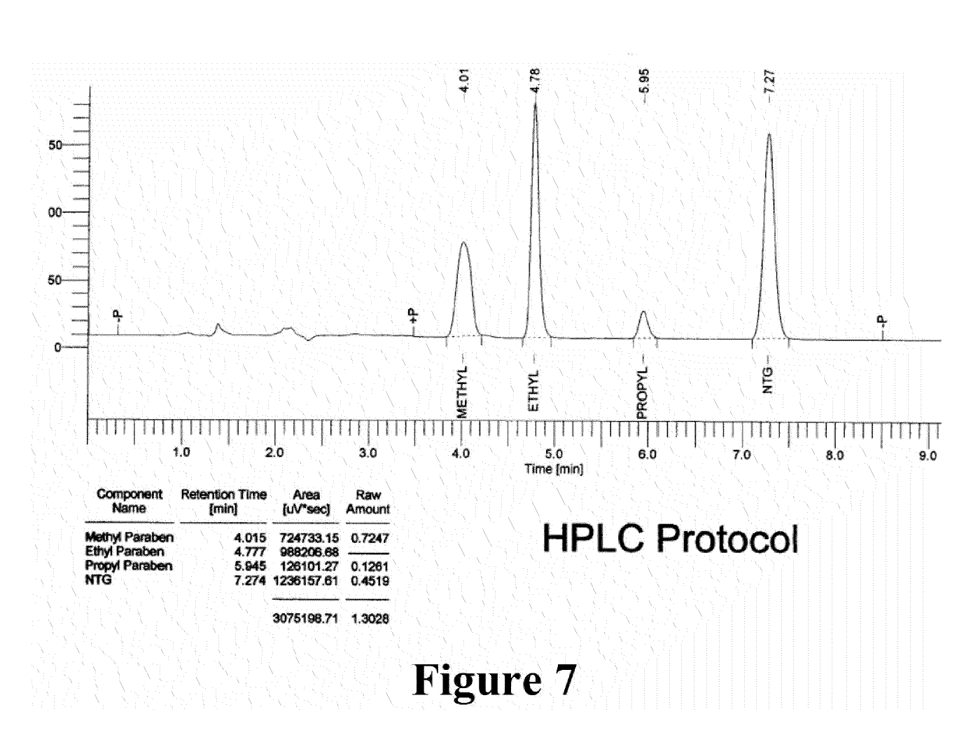

Samples are taken out of incubation at 4-5° C. and 38-40° C. and analyzed at room temperature by HPLC. Ethyl paraben is employed as an internal standard and sample is run at 214 nm wavelength on C18 column with water and acetonitrile as mobile phases. An exemplary HPLC chromatogram of a sample analyzed by the disclosed protocol is shown in FIG. 7. In that example, a pH 5.5 cream containing 0.6% nitroglycerin cream and subjected to accelerated temperature of 38-40° C. for 4.5 months, showed no appreciable decomposition of nitroglycerin.

b. pH Measurements

Samples are taken out of incubation from 4-5° C. and 38-40° C. and measured at room temperature for pH. A pH meter suitable for measuring creams is used after it is first calibrated using buffer solutions at pH 4.01, pH 7.0, and pH 10.01

c. Viscosity Measurements

Samples are removed from environments held at constant 4-5° C. and 38-40° C. and measured at room temperature for viscosity using a Brookfield viscometer equipped with a S06 or S07 spindle; measurements are made at 0.5 rpm and 1.0 rpm. It is understood that the same physical sample can be used for both the pH and the viscosity measurement.

2. General Method for making Stable Water-Based Topical Pharmaceutical Creams

Methyl paraben, propyl paraben, isopropyl palmitate, polyethylene glycol and propylene glycol were added to a vessel, and the mixture was stirred. Preheated or cold water was added to this solution and then Carbopol was slowly added, with continued stirring, followed by the addition of methylcellulose. The resulting mixture was stirred and temperature maintained at 60° C. until a uniform suspension was observed, followed by the addition of Brij or Tween 80. A 10% aqueous triethanolamine solution was then slowly added and pH was subsequently adjusted to between about 5 to about 5.3 by adding triethanolamine as needed. The resulting gel was weighed, and additional water was added if evaporation occurred.

When the temperature of the gel reached between about 30° C. and about 35° C., a solution of nitroglycerin/propylene glycol was added slowly with stirring. A pH reading was then taken to ensure that the appropriate pH was maintained.

It is understood that the nitroglycerin amount in the cream can be varied by varying the amount of propylene glycol in the nitroglycerin/propylene glycol solution. For example, a mixture of about 30 g of 10% nitroglycerin (in propylene glycol) can be mixed with about 70 g of propylene glycol to provide a cream comprising about 0.3% nitroglycerin. About 90 g of 10% nitroglycerin (in propylene glycol) can be used with 10% additional propylene glycol to provide a cream comprising about 0.9% nitroglycerin. For concentrations above 1% nitroglycerin, extra propylene glycol is typically not added, because nitroglycerin is typically commercially available as a 10% solution in propylene glycol.

It should also be appreciated that an aqueous triethanolamine solution can be prepared as a stock aqueous solution (e.g., 10%, 30%, etc. in aqueous solvent) in advance (e.g., days before the final formulation is made) and used analogously in proportional amounts. Triethanolamine can also be used undiluted.

Table 4 provides an exemplary composition for a 1000 g batch of cream comprising about 0.6% nitroglycerin.

TABLE 4
EXEMPLARY COMPOSITION FOR 1000
G OF 0.6% NITROGLYCERIN CREAM

| Ingredient | Amount/g | Wt. % |
|---|---|---|
| Methyl Paraben | 0.8 | 0.08 |
| Propyl Paraben | 0.2 | 0.02 |
| Isopropyl Palmitate | 20.0 | 2.00 |
| Propylene Glycol | 40.0 | 4.00 |
| Water | 859.0 | 85.9 |
| Carbopol 934P | 7.0 | 0.70 |
| Methyl Cellulose | 4.0 | 0.40 |
| Tween 80 | 5.0 | 0.50 |
| Triethanolamine | 4.0 | 0.4 |
| 10% nitroglycerin in propylene glycol | 60.0 | 6.00 |

3. Determination of Cream Thermal Stability

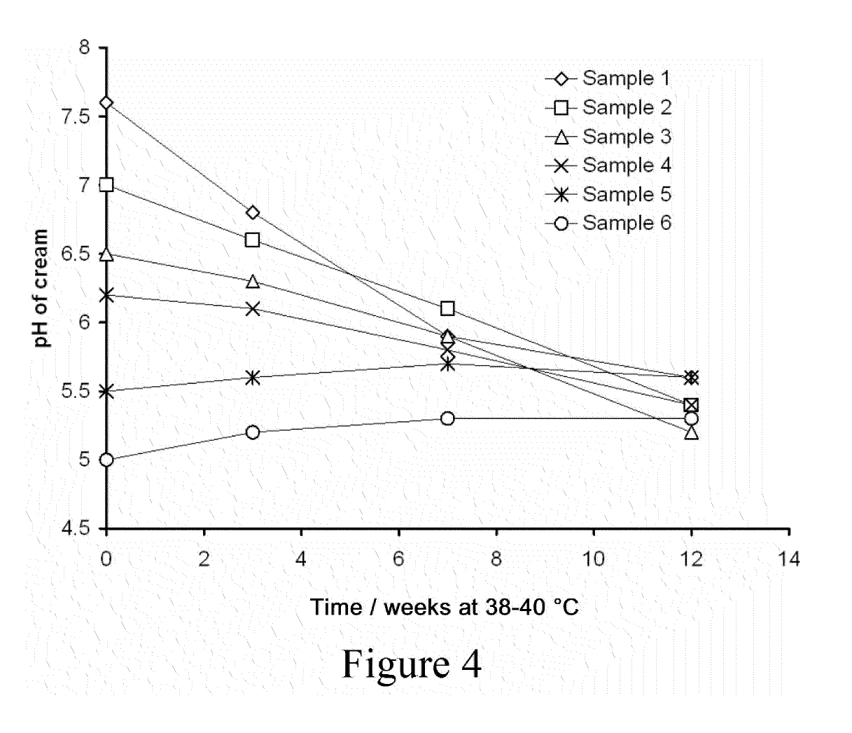

Each cream comprising nitroglycerin was made according to the methods described in the previous example. Multiple sample creams were made, each having a distinct initial pH. In an example experiment, samples 1-6 were made with initial pH values of 7.6, 7.0, 6.5, 6.2, 5.5 and 5.0 respectively. Each sample was incubated at about 38-40° C., and analyzed at room temperature at various time intervals (e.g. 0, 3, 7, and 12 weeks) using pH and HPLC. FIG. 4 shows the pH values of the samples at the various time intervals. The results indicate that a higher initial pH value of cream typically provides a final sample with less nitroglycerin in the composition. Table 5 shows a comparison of pH and HPLC analysis done at room temperature of the same samples incubated for 12 weeks at 38-40° C. An initial HPLC peak area of 100% for nitroglycerin at time=0 weeks was used as a reference value.

TABLE 5
PH AND HPLC RESULTS AFTER 12 WEEKS AT 38-40° C.

| Sample | Initial pH | pH after 12 weeks at 40° C. | Final peak area |
|---|---|---|---|
| 1 | 7.6 | 5.4 | 74% |
| 2 | 7.0 | 5.5 | 79% |
| 3 | 6.5 | 5.2 | 88% |
| 4 | 6.2 | 5.4 | 91% |
| 5 | 5.5 | 5.6 | 94% |
| 6 | 5.0 | 5.3 | 97% |

4. Relationship between Viscosity, pH, and Stability

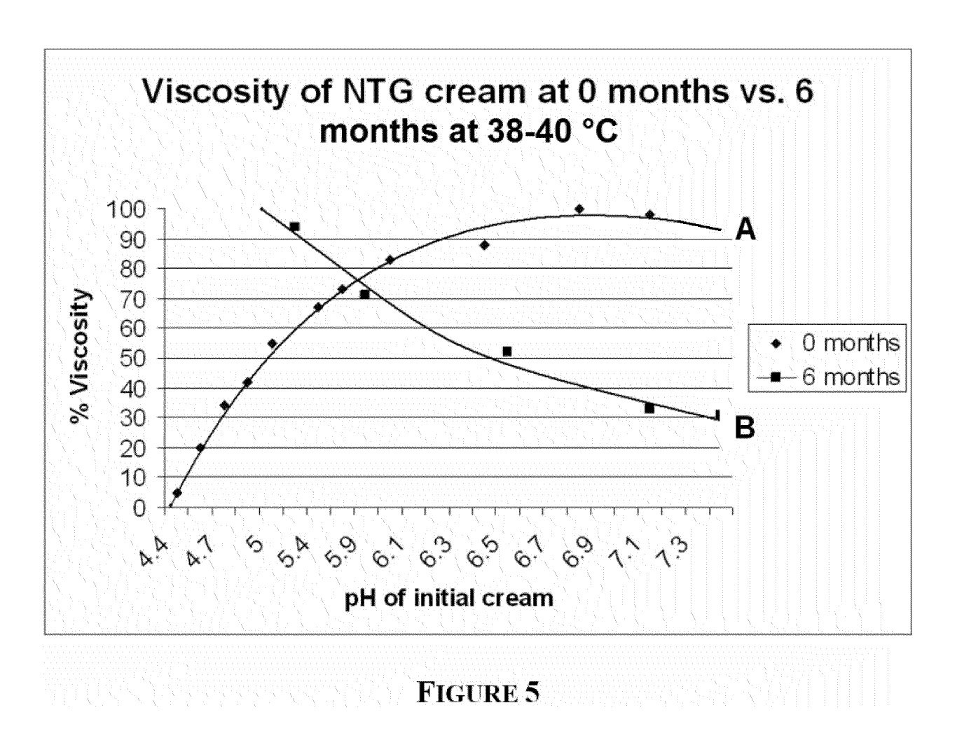

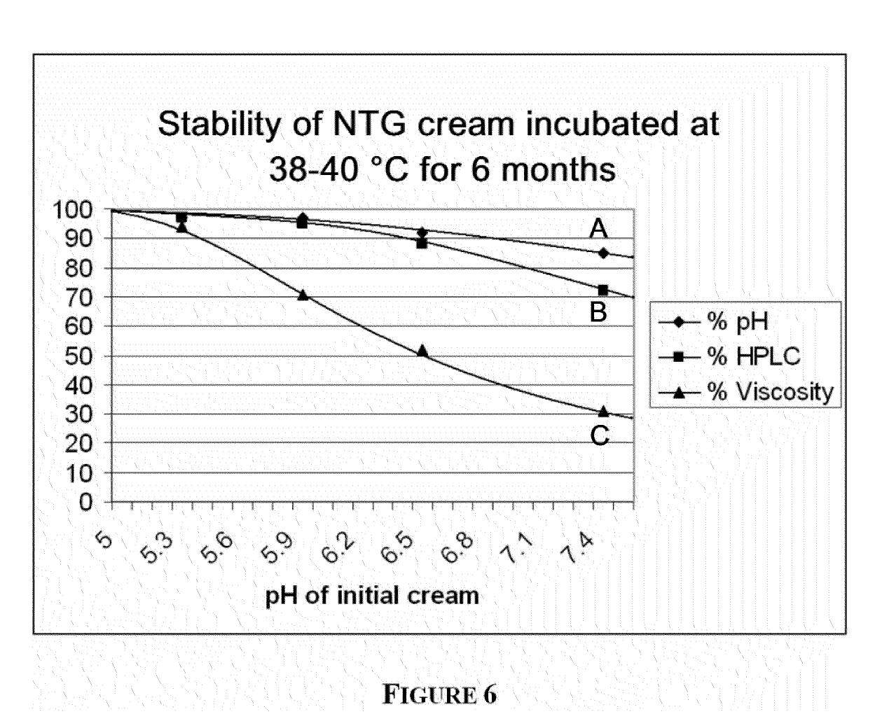

To determine the relationship between pH, stability, and viscosity (q) experiments were carried out using varying initial pH creams prepared according to the general method for making stable water-based topical pharmaceutical creams. Viscosity readings were then recorded at room temperature as intial pH of creams was increased, and readings were graphed as % difference from the highest viscosity reading that was achieved around pH 7, shown in FIG. 5, curve A. Curve B shows the deterioration in viscosity of creams that were prepared at different intial pHs that have been incubated at 38-40° C. for 4.5 months. Curve B was graphed using viscosity readings of same creams incubated at 4-5° C. (taken as 100%) and compared to creams incubated at 38-40° C. FIG. 6 shows a graphical representation of Table 6 for the creams prepared at different initial pH after being incubated for 6 months at 38-40° C. using three types of analysis measured at room temperature. Curve A shows the % drop in pH after 6 months at 38-40° C. (cream at 4-5° C. taken to be 100%). Curve B shows the % drop in HPLC peak area for nitroglycerin (cream at 4-5° C. taken to be 100%). Curve C shows the % drop in viscosity of cream after 6 months at 38-40° C. (cream at 4-5° C. taken to be 100%).

TABLE 6
SIX-MONTH STABILITY RESULTS AFTER
INCUBATION AT 38-40° C.

| pH | % pH | % HPLC Peak area | % viscosity |
|---|---|---|---|
| 5.3 | 97% | 97% | 94% |
| 5.9 | 97% | 95% | 71% |
| 6.5 | 92% | 88% | 52% |
| 7.1 | 88% | 78% | 33% |
| 7.4 | 85% | 72% | 31% |

5. Treatment of Peripheral Vascular Disease (Prophetic)

Using known techniques (e.g., diagnosis by a medical professional), a mammal (e.g., a human) in need for treatment for peripheral vascular disease can be identified. A therapeutically effective amount (e.g., a thin coating of the cream applied at the affected areas of the mammal) of the disclosed water-based topical creams comprising nitroglycerin can be topically administered to the mammal.

At intervals during the treatment regimen, the mammal is monitored for symptoms of peripheral vascular disease. After the treatment period has concluded, again using known techniques, the mammal is found to have decreased symptoms of peripheral vascular disease and/or to be no longer in need for treatment for peripheral vascular disease.

6. Chemotherapy and Co-Adminstration of Water-Based Topical Creams Comprising Nitroglycerin for Treatment of Drug-Induced Insufficient Circulation (Prophetic)

A mammal (e.g., a human) can be treated with chemotherapy—which is known to be associated with decreased circulation—thereby resulting in insufficient circulation. Thus, using known techniques (e.g., diagnosis by a medical professional), the mammal can be identified as in need for treatment for drug-induced insufficient circulation. A therapeutically effective amount (e.g., a thin coating of the cream applied at the affected areas of the mammal) of the disclosed water-based topical creams comprising nitroglycerin can be topically co-adminstered with the chemotherapy to the mammal, thereby alleviating the insufficient circulation. After the chemotherapy treatment has concluded, again using known techniques, the mammal is found to have decreased symptoms of insufficient circulation and/or to be no longer in need for treatment for insufficient circulation.

Alternatively, a prophylactically effective amount (e.g., a thin coating of the cream applied at the affected areas of the mammal) of the disclosed water-based topical creams comprising nitroglycerin can be topically adminstered to the mammal before, during or after the chemotherapy, thereby preventing or alleviating the insufficient circulation.

7. Treatment of Diabetes and Co-Administration of Water-Based Topical Creams Comprising Nitroglycerin for Treatment of Disease-Induced Insufficient Circulation (Prophetic)

A mammal (e.g., a human) can be treated for diabetes—a disorder associated with insufficient circulation—with a drug (e.g., insulin) known to be effective for treating the diabetes, but ineffective for alleviating insufficient circulation stemming from the diabetes, thereby resulting in insufficient circulation in the mammal. Thus, using known techniques (e.g., diagnosis by a medical professional), the mammal can be identified as in need for treatment for disease-induced insufficient circulation. A therapeutically effective amount (e.g., a thin coating of the cream applied at the affected areas of the mammal) of the disclosed water-based topical creams comprising nitroglycerin can be co-adminstered with the drug known to be effective for treating insufficient circulation to the mammal, thereby alleviating the insufficient circulation. After the adminstration has concluded, again using known techniques, the mammal is found to have decreased symptoms of insufficient circulation and/or to be no longer in need for treatment for insufficient circulation.

8. Enhancing Localized Activity of an NSAID (Prophetic)

A mammal (e.g., a human) can be treated for muscular (e.g., back muscles) inflammation with a non-steroidal anti-inflammatory drug (NSAID) (e.g., ibuprofen)—known to be systemically effective for treating inflammation—for a localized muscular inflammation. Thus, using known techniques (e.g., diagnosis by a medical professional), the mammal can be identified as in need for treatment for muscular inflammation. A therapeutically effective amount (e.g., a thin coating of the cream applied at the affected areas of the mammal) of the disclosed water-based topical creams comprising nitroglycerin can be applied to the specific external surface area of a mammal and co-adminstered with the NSAID, thereby enhancing the desired systemic effect proximate to the external surface area of the mammal. After the adminstration has concluded, again using known techniques, the mammal is found to have decreased symptoms of muscular inflammation and/or to be no longer in need for treatment for muscular inflammation.

9. Enhancing Circulation (Prophetic)

An effective amount (e.g., a thin coating of the cream applied at the affected areas of the mammal) of the disclosed water-based topical creams comprising nitroglycerin can be topically administered to an external surface area of a mammal, thereby increasing circulation in the vasculature proximate to the external surface area of the mammal to a level greater than that of the external surface area prior to administration. After adminstration, again using known techniques, the mammal is found to have increased circulation in the vasculature proximate to the external surface area of the mammal to a level greater than that of the external surface area prior to administration.

Alternatively, an effective amount of one or more nutrients (e.g., vitamins and/or moisturizers) can be topically co-administered to the external surface area of the mammal before, during, or after topical administration of the disclosed water-based topical creams comprising nitroglycerin.

10. Wound Healing (Prophetic)

Using known techniques (e.g., diagnosis by a medical professional), a mammal can be identified as in need for treatment for wound healing. A therapeutically effective amount (e.g., a thin coating of the cream applied at the affected areas of the mammal) of the disclosed water-based topical creams comprising nitroglycerin can be applied to the specific external surface area of the wound of the mammal, thereby increasing hemodynamic blood flow into the injured tissue and thus promoting wound healing relative to an otherwise substantially identical wound that is not treated with topical administration of the disclosed water-based topical creams comprising nitroglycerin. After the treatment regimen has concluded, again using known techniques, the wound is found to be decreased in severity and/or the mammal is no longer in need for treatment for the wound.

11. Example Manufacturing Procedure

The order of ingredient addition can be the same as outlined in Table 7. All ingredients are added to large stainless steel mixing bowl equipped with a variable mixing motor with a propeller stirrer capable of holding at least 100 kg of product.

TABLE 7
BATCH FORMULATION (IMX-150-0808-E)

| Formula | 1.2% NTG | Weight (g) |
|---|---|---|
| pH Target | pH 5.2 | n/a |
| Total Weight | 8 Kg | 8,000.0 |

| | % Formula | Weight (g) |
|---|---|---|
| Methyl Paraben | 0.08% | 6.4 |
| Propyl Paraben | 0.02% | 1.6 |
| PEG 8000 | 0.50% | 40.0 |
| Isopropyl Palmitate | 2.00% | 160.0 |
| Water | 83.45% | 6,676.0 |
| carbopol 934P | 0.70% | 56.0 |
| Methyl Cellulose | 0.40% | 32.0 |
| Polysorbate 80 | 0.50% | 40.0 |
| Triethanolamine (TEA) | 0.35% | 28.0 |
| 10% Nitroglycerin | 12.00% | 960.0 |

First, weigh methyl paraben and add to bowl. Next, weigh propyl paraben and add to bowl. Next, weigh PEG8000 and add to bowl. Then, weigh isopropyl palmitate and add to bowl. Next, swirl ingredients or mix lightly so that the dry ingredients are uniformly distributed in the liquid. Then, add heated (70-80° C.) WFI water to bowl and start stirring with propeller blade. Next, weigh Carbopol 934P and slowly add to bowl while stirring and keep stirring until all is uniformly distributed. Then, weigh Methyl Cellulose and slowly add to bowl while stirring and keep stirring until all is uniformly distributed. Next, weigh Polysorbate 80 and slowly add to bowl while stirring. Then, stir for 10 minutes. Next, change propeller stirrer to a mixer blade and continue stirring. Then, add triethanolamine and stir for 15 minutes.

Next, take pH reading. pH should be at 5.1±0.1 (range of pH 5.0-5.2). If base cream is below pH 5.0, add more TEA, stir for 15 minutes and recheck pH reading to achieve a pH of 5.1±0.1. An acceptable pH range is 5.0 to 5.4. Then, add appropriate amount of 10% nitroglycerin in propylene glycol. Stir for 30 minutes. Next, take pH reading after nitroglycerin has been added. pH should be pH 5.2±0.2 for a range of pH 5.0-5.4. If pH is below 5.0, add more TEA, stir 30 minutes and take another reading until a pH of 5.0-5.4 achieved.

The resulting nitroglycerin creams showed excellent stability over time, as tabulated below for incubation at 25° C. and 40° C., respectively.

Tables 8A and 8B. Stability of IMX-150 1.2% Nitroglycerin

8A: Incubation at 25° C.

| Months | pH | % Nitroglycerin |
|---|---|---|
| 0 | 5.1 | 94.3 |
| 1 | 5.3 | 101.7 |
| 3 | 5.2 | 95.8 |
| 6 | 5.2 | 106.2 |

8B: Incubation at 40° C.

| Months | pH | % Nitroglycerin |
|---|---|---|
| 0 | 5.1 | 94.3 |
| 1 | 5.2 | 99.4 |
| 2 | 5.2 | 99.7 |
| 3 | 5.2 | 97.9 |
| 6 | 5.1 | 108.2 |

12. Multiple-Vessel Preparation of a Water-Based Topical Cream Containing Nitroglycerin (Comparative Example)

The topical creams are prepared by admixing a thickener and about 80% to about 90% of water portion and heating to a temperature of about 45° C. to about 75° C. to provide a first solution. Admixing an emulsifier, a penetration enhancer, auxiliary ingredients, pH adjustor and the remaining portion of the water with heating to a temperature of about 35° C. to about 70° C. results in a second solution. Next, admixing the first solution and second solution at a lowered temperature, preferably below about 50° C., provides a cream base. Admixing nitroglycerin, remaining portion of the penetration enhancer, and any auxiliary ingredients and the cream base, in propylene glycol results in a water-based topical cream containing nitroglycerin. These creams preferably have a pH value of about 6.5 to about 9.0. Creams prepared by this procedure exhibit an accelerated nitroglycerin and cream base decomposition measurement profile substantially inferior to the disclosed inventive creams. Creams prepared by this procedure are more complex to make and appreciably more time consuming.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A water-based topical cream comprising:
a. nitroglycerin;
b. one or more penetration enhancers;
c. at least about 60% of an aqueous solvent by weight of the cream; and
d. one or more pH-adjusters in an amount sufficient to provide pH of the cream at from about 4 to about 6.

2. The cream of claim 1, wherein the one or more pH-adjusters is present in an amount of from about 0. 1% to about 1% by weight of the cream.

3. The cream of claim 1, wherein the one or more pH-adjusters is a base selected from hydroxides, carbonates, ammonia, amines, borates, phosphates, and citrates.

4. The cream of claim 3, wherein the base is an amine selected from triethylamine, diethylmethylamine, ethyldimethylamine, triethanolamine, and isopropyldimethylamine.

5. The cream of claim 1, wherein the one or more penetration enhancers are present as from about 1% to about 10% by weight of the cream.

6. A method for preparing a water-based topical cream, the method comprising the steps of:
a. providing a mixture of an aqueous solvent, one or more penetration enhancers, and, optionally, one or more preservatives;
b. adding to the mixture one or more thickeners;
c. adding to the mixture one or more emulsifiers;
d. heating the mixture to at least a temperature sufficient to melt or solubilize the one or more thickeners and/or one or more emulsifiers; and
e. adjusting the pH of the mixture to from about 4 to about 6 with a pH-adjuster.

7. The method of claim 6, wherein the steps are performed in a single vessel.

8. The method of claim 6, further comprising the step of adding a pharmaceutically active ingredient.

9. The method of claim 8, wherein the pharmaceutically active ingredient comprises nitroglycerin.

10. The method of claim 6, wherein the one or more pH-adjusters is present in an amount of from about 0.1% to about 0.7% by weight of the cream.

11. The method of claim 6, wherein the one or more pH-adjusters is a base selected from hydroxides, carbonates, ammonia, amines, borates, phosphates, and citrates.

12. The method of claim 11, wherein the base is an amine selected from triethylamine, diethylmethylamine, ethyldimethylamine, triethanolamine, and isopropyldimethylamine.

13. A method of treating a circulatory disorder comprising topically administering to a mammal a therapeutically effective amount of water-based topical cream comprising nitroglycerin and at least about 60% by weight of an aqueous solvent and having a pH of from about 4 to about 6, thereby treating the disorder in the mammal.

14. The method of claim 13, wherein the mammal is human.

15. The method of claim 13, wherein the mammal has been diagnosed with the circulatory disorder prior to administration.

16. The method of claim 13, further comprising the step of identifying a mammal in need of treatment of the circulatory disorder.

17. The method of claim 13, wherein the disease is one or more of peripheral vascular disease, peripheral artery disease, male impotence, Raynaud's disease, diabetic peripheral neuropathy, vulvodynia, female anorgasmia, anal fissues, coronary artery disease, nocturnal leg cramps, restless leg syndrome, and pressure wounds.

18. A method of promoting wound healing comprising topically administering at or proximate to injured tissue of a mammal an effective amount of water-based topical cream comprising nitroglycerin and at least about 60% of an aqueous solvent by weight of the cream, thereby increasing hemodynamic blood flow into the injured tissue.

19. The method of claim 18, wherein the hemodynamic blood flow is increased by at least about 10% after administration.

20. The method of claim 18, wherein the injury is a bruise, a laceration, an abrasion, a burn, or frostbite.
